(19) United States
(12) Patent Application Publication
(10) Pub. No.: US 2008/0251374 A1
(43) Pub. Date: Oct. 16, 2008

(54) SYSTEM AND METHOD FOR PRODUCING ETHANOL FROM PAPER MILL SLUDGE

(75) Inventor: Brent Alan McManigal, Cherry Tree, PA (US)

Correspondence Address: MCNEES WALLACE & NURICK LLC, 100 PINE STREET, P.O. BOX 1166, HARRISBURG, PA, 17108-1166, US

(73) Assignee: TEAM TEN, LLC, Tyrone, PA (US)

(21) Appl. No.: 11/735,633

(22) Filed: Apr. 16, 2007

(51) Int. Cl. C07C 29/80

(52) U.S. Cl. 203/19

(57) ABSTRACT

A method and system are disclosed for converting paper mill sludge into ethanol. The method and system include selecting effluent streams within the paper mill having high BOD levels to combine with the sludge to both facilitate the treatment of the sludge and contribute effluent BOD to the production of ethanol.

### FIELD OF THE INVENTION

The present invention is directed to a system and method of producing ethanol from paper mill sludge materials and effluent streams.

### BACKGROUND OF THE INVENTION

Paper products are the largest component of municipal solid waste, making up to 31-38% of the composition of landfills in the United States. In 2005, 51.5 percent of the paper consumed in the U.S. was recovered for recycling. This means that today, in 2007, over 51 million tons of paper and paper products are being recovered for recycling annually. The U.S. paper industry has set a goal to recover 55 percent of all the paper consumed in the U.S. by 2012.

Paper recycling is the process of converting waste paper or scrap paper into a usable product. The term “paper recycling” could refer to burning waste paper for energy, but it typically refers to converting waste paper back into a useable product. This typically includes: collection, mechanical conversion of the waste paper to mulch, mixing the mulch with water to create a slurry, chemically treating the slurry to separate the paper fibers, chemically bleaching the slurry to whiten the product, and extracting the remaining chemical slurry to form new sheets of paper.

A waste paper treatment process for converting waste paper into a pulp suitable for re-use will often include one or more operations designed, for example, to separate printing ink from the cellulosic fibers in the waste paper. Generally in a plant for de-inking waste paper, the waste paper is first pulped. For example, pulping may take place in a pulper provided with a low attrition rotor in water containing various chemical reagents, and at a consistency which is typically in the range from about 8% to about 18% by dry weight of waste paper. The chemical reagents detach the ink particles from the fibers and render the particles hydrophobic. The pulper is generally operated at a temperature in the range from about 90° F. to 130° F. and at pH between about 6 to about 9. On completion of the pulping operation, the pulp is usually diluted with water to about 1% to about 2% by weight pulp. Additional pulper and screen flow streams may be present to allow for the treatment of different grades of waste paper, such as waste paper that may not need to be de-inked.

The pulp suspension is then passed to a system of mechanical separators that remove relatively heavy foreign bodies, such as staples, paper clips and particles of grit, and also lightweight contaminants, such as plastics materials, and glue clumps that arise from adhesives used in paper coating, binding or laminating, and from hydrophobic reagents, such as sizing agents. The suspension passing through the separators is then fed to a treatment plant that usually comprises one or more froth floatation cells, or one or more washing units, or a combination of floatation cells and washing units.

For example, a typical treatment might comprise one or more froth floatation cells, each of which includes a means to introduce air in the form of fine bubbles. Air bubbles attach to the hydrophobic ink particles and lift the particles to the surface of the suspension. Ink particles having sizes in the range from about 20 microns to about 200 microns are concentrated in the froth that overflows from the top of the floatation cells, while a pulp suspension depleted in ink is withdrawn from the bottom. The pulp suspension from the bottom of the floatation cells is then passed through additional mechanical separators to remove any small contaminants and finally dewatered to produce a substantially de-inked pulp that can be further processed into a paper product.

The water recovered from the dewatering operations is generally cloudy as a result of its content of fine particulate material that may comprise, for example, inorganic filler particles or organic particles such as ink of fine fibers. It is necessary to remove as much as possible of this particulate material before this water is suitable for recycling for re-use in earlier stages of the de-inking process. The cloudy water may be cleaned, for example in microfloatation cells in which fine bubbles of air are injected at the base of the cells and the particulate material is carried upwards by air bubbles that attach themselves thereto.

The treatment plant reject solids and streams, which are usually in the form of a suspension, or slurry, include the froth product from any floatation stages and/or the suspension that has passed through screens during any washing stage. These suspensions generally contain, in addition to the ink particles, a substantial proportion of the cellulose fibers and inorganic filler or pigment particles that were originally present in the waste paper. The filler particles consist predominantly of a mixture of kaolin clay and calcium carbonate in various proportions, although other inorganic filler particles such as talc, calcium sulphate or titanium dioxide may also be present in minor amounts.

The reject or effluent stream carrying a high solids load from the waste paper de-inking plant is generally known as “sludge”. This sludge includes particulates, such as fillers, inking materials such as carbon and various organic inks, other organic substances such as lattices and other adhesives, sizing agents, and the like. The organic substances may also include cellulose, lignin and hemicellulose. The sludge is generally a sticky, highly discolored or gray mass. The water of the sludge contains a variety of soluble substances that reduce the usability of the water. This sludge has traditionally been disposed of by landfilling, composting, incorporation into cement, and incineration. Thus, the useful organic material contained in the sludge has for the most part been wasted or used as a source of very low grade fuel.

Another byproduct of the waste paper treatment process is the effluent or waste liquid streams. These effluent streams are high in dissolved and suspended organic components generated during various stages of the waste paper treatment process. Often, these effluents are too contaminated with organic and inorganic material to be recycled in the process. Sometimes, the effluent may be subject to separation to allow for a part of the effluent to be recycled, but this only results in a stream that is even higher in organic and inorganic material being produced that must be further treated. The effluent streams create a difficult disposal and processing problem for waste paper recyclers because of the large volume of the steams and the environmental disposal concerns associated with the streams.

The conversion of both sludge and effluent into a useful product has long been desired. However, the mineral content of the sludge and the volume of effluent has made it impractical to develop a cost effective use for these waste materials. Thus, a need exists to find a useful application for the byproduct waste material from the waste paper treatment process.

At the same time, another need exists to find an alternative fuel source to the limited amount of gasoline available today. Ethanol has been proposed as both a supplement and alternative to gasoline. Ethanol has been produced from biomass sources high in cellulose such as corn and other biodegradable products. The biomass source is treated to form sucrose that is further processed into ethanol. However, this type of biomass has other useful applications including food and animal feed.

Up to this time, no practical scheme has been proposed to convert papermaking byproduct waste into ethanol. Therefore, a need also exists to convert waste byproducts from the paper making process into useful ethanol at a reasonable cost.

### SUMMARY OF THE INVENTION

Accordingly, it is an object of the present invention to provide a cost effective and practical process to convert the waste paper manufacturing process byproducts of sludge and effluent steams into ethanol. The invention provides for a predetermined combination of selected effluent streams and sludge to form a slurry that is effectively converted into ethanol.

According to a first embodiment of the invention, a method for producing ethanol is provided that includes providing an amount of waste paper process sludge, combining the sludge with a selected waste paper process effluent stream to form a slurry containing cellulose, adding a reducing agent to the slurry, adding cellulose enzymes to the slurry, cooking the slurry to convert a substantial portion of the cellulose to sucrose, separating the cooked slurry into a liquid portion and a solids portion, filtering the liquid portion to form a filtered liquid portion, adding yeast to the filtered liquid portion, fermenting the filtered liquid portion to form a beer whereby a substantial portion of the sucrose has been converted to ethanol, distilling the beer to form an ethanol stream, and passing the ethanol stream through a molecular separator to remove water from the ethanol stream and form substantially pure ethanol.

The first embodiment further includes wherein the selected effluent stream includes a de-inking system discharge, and wherein the selected effluent stream further includes whitewater from a paper machine process and starch losses, and wherein the selected effluent stream has a BOD of between about 500 mg/l and about 1100 pppm. The first embodiment also includes wherein the reducing agent is ammonia, and wherein the molecular separator is a molecular sieve, and further includes wherein the liquid portion is cooled before adding the yeast, and wherein the cooking takes place at a temperature of between about 140° F. for between about 16 hours to about 24 hours, and wherein the fermenting takes place for about 48 hours.

Another embodiment of the invention provides for a method for producing ethanol from paper mill sludge that includes providing an amount of paper mill sludge, combining the paper mill sludge with a selected paper mill effluent stream to form a slurry containing cellulose, adding additional sources of cellulose to the slurry, adding ammonia to the slurry, adding cellulose enzymes to the slurry, cooking the slurry to convert a substantial portion of the cellulose to sucrose; separating the cooked slurry into a liquid portion and a solids portion, filtering the liquid portion, adding yeast to the filtered liquid portion, fermenting the filtered liquid portion to form a beer whereby a substantial portion of the sucrose has been converted to ethanol, distilling the beer to form an ethanol stream, and passing the ethanol stream through a molecular sieve to remove water from the ethanol stream and form substantially pure ethanol.

This embodiment of the invention further includes wherein the slurry is cooked at temperature of about 140° F. for between about 16 hours to about 24 hours to convert the substantial portion of the cellulose to sucrose.

Another embodiment of the invention provides for a system for producing ethanol that includes providing a source of paper mill sludge containing cellulose, a selected effluent stream containing dissolved organic compounds, a reactor for receiving the sludge and the liquid steam and forming a slurry, a source of cellulose enzymes for providing cellulose enzymes to the slurry, a second reactor for receiving the slurry, a heater for heating the second reactor to a temperature of about 140° F., a solids removal line for removing solids from the second reactor, a pipe for removing liquid from the second reactor, a fermentation tank connected to the liquid removal line for receiving the liquid removed from the second reactor, a source of yeast for supplying yeast to the fermentation tank, a pipe for removing fermented liquid from the fermentation tank, a distillation tower for receiving the fermented liquid from the pipe, a heat source for heating the distillation tower, a pipe for removing an ethanol mixture from the distillation tower, a molecular sieve attached to the pipe removing the ethanol mixture, and a pipe that removes a substantially pure ethanol stream from the molecular sieve.

This embodiment of the invention further includes wherein the selected effluent stream comprises a selected waste paper mill effluent stream, and also includes wherein the selected waste paper mill effluent stream has a BOD of between about 500 ppm and about 1100 ppm.

Yet another embodiment of the invention provides for a system for converting waste paper mill sludge into ethanol, the system including a source of paper mill sludge containing cellulose, a selected paper mill effluent stream containing dissolved organic compounds, a reactor for receiving the sludge and the liquid stream and forming a slurry, a source of cellulose enzymes for providing cellulose enzymes to the slurry, a second reactor for receiving the slurry, a heater for heating the second reactor to a temperature of about 140° F., a solids removal line for removing solids from the second reactor, a pipe for removing liquid from the second reactor.

The above embodiment additionally includes a fermentation tank connected to the liquid removal line for receiving the liquid removed from the second reactor, a source of yeast for supplying yeast to the fermentation tank, a pipe for removing fermented liquid from the fermentation tank, a distillation tower for receiving the fermented liquid from the pipe, a heat source for heating the distillation tower, a pipe for removing an ethanol mixture from the distillation tower, a molecular sieve attached to the pipe removing the ethanol mixture, and a pipe that removes a substantially pure ethanol stream from the molecular sieve.

The above embodiment of the invention further includes at least one other source of cellulose added to the paper mill sludge, and further includes at least one other selected effluent stream containing dissolved solids added to the paper mill liquid stream.

The disclosed invention aims to solve the above problems by using byproduct waste from a paper making process as a source of biomass for the production of ethanol.

Further aspects of the method and system are disclosed herein. The features as discussed above as well as other features and advantages of the present invention will be appreciated and understood by those skilled in the art from the following detailed description and drawings.

### BRIEF DESCRIPTION OF THE DRAWINGS

FIG. 1 is a flow chart of paper production in a waste paper mill facility.

FIG. 2 is a schematic of an exemplary waste paper mill effluent system.

FIG. 3 is a flow chart of an exemplary ethanol production method.

### DETAILED DESCRIPTION OF THE INVENTION

Disclosed herein is an example of a system and method for producing ethanol from waste paper mill waste sludge and effluent streams. The disclosed system and method combines paper mill sludge with selected high BOD effluent streams to form a slurry that is then processed to form ethanol.

A typical mid-sized paper making plant processing about 2000 tons per week of waste paper may produce about 1700 tons/week of wastepaper and 300 tons per week of sludge. The same plant may produce about 2.2 million gallons of liquid waste effluent per day. The sludge is mainly composed of cellulose and entrained minerals. The waste effluent contains dissolved organic material, mostly starch. The amount of dissolved organic material is measured by its biological oxygen demand (BOD). BOD is a measure of the amount of oxygen required by aerobic microorganisms to decompose the organic matter in a sample of water, and thus is a good indicator as to the amount of total dissolved organic matter in a liquid.

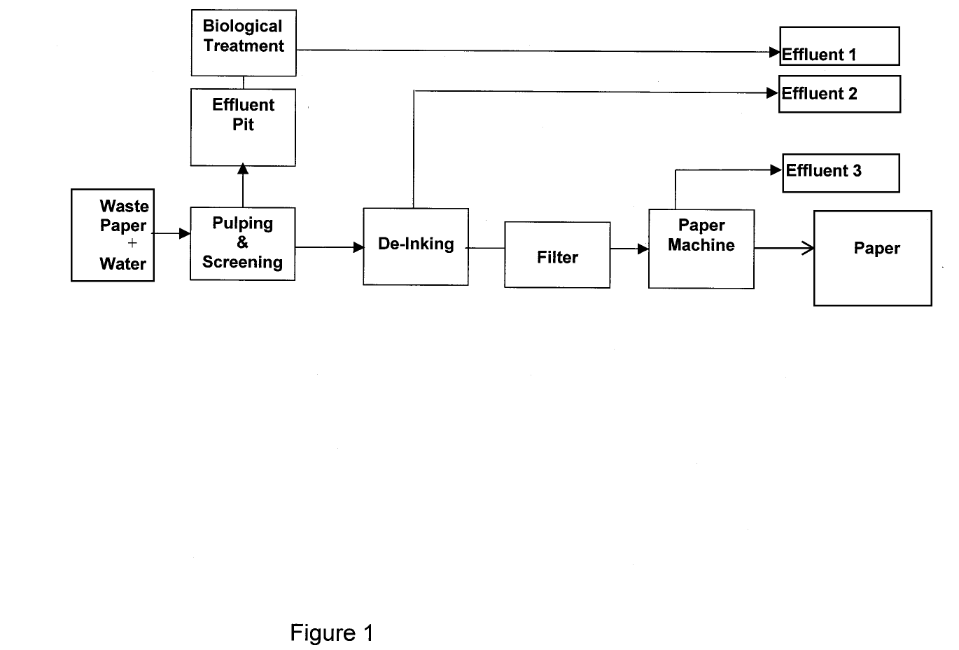

A flow chart of a paper production at an exemplary waste paper process facility is shown at FIG. 1. As can be seen in FIG. 1, waste paper is provided to a pulping station where the waste paper is mixed with water and chemicals to form a pulp mixture. The pulp mixture is agitated in a pulper to form waste paper pulp suspension of cellulose fibers in water. The water may be fresh water, referred to as make-up water, recycled water from other plant operations, or a combination of fresh and recycled water. The recycled water may contain dissolved and suspended components, including starch and cellulose, and may be referred to as whitewater. The pulper contains a rotor or impeller to vigorously agitate the waste paper and water to form the suspension. The pulper is operated in at temperature of about 90° F. to about 130° F. and at a pH of about 6 to about 9. The suspension may contain about 4% to about 5% by weight of dry solids, which provides for suitable slurry to be pumped to subsequent treatment stations.

The waste paper pulp suspension is then provided to mechanical separators and/or screens where relatively heavy foreign bodies, such as staples, paper clips, and particles of grit, and also lightweight contaminants, such as plastics materials and “stickies,” which arise from adhesives used in paper coating, binding or laminating, and from hydrophobic reagents, such as sizing agents are separated. The mechanical separators may be a single coarse separator or a series of screen separators as required by the degree of contamination of the initial waste paper.

The foreign bodies are thus separated, washed with water, and provided to an effluent pit. The effluent wash water is recovered and the solids removed by screening and floatation. The effluent wash water may then be provided to a collective waste effluent stream. The collective waste effluent stream may be a piping system that runs throughout the process facility to collect liquid effluent streams for later processing. The wash water may be fresh water, whitewater, or a combination of fresh and whitewater.

Effluent wash water is recovered from the effluent pit and may be provided to a biological treatment process or supplied to the ethanol production stages as Effluent 1. Effluent 1 is a high BOD effluent stream with a BOD of between about 500 ppm and 1100 ppm. A waste paper mill processing approximately 250 tons of waste paper per day would produce approximately 0.15 million gallons per day (mgpd) of Effluent 1.

It should be understood that more than one waste paper feed, most often of varying quality of waste papers, may be provided to more than one pulper to form the waste paper pulp. In this case, waste papers having very small amounts of foreign bodies may require very little, if any, mechanical separation and/or screening in the pulping process.

The waste paper pulp is then passed through additional mechanical separators and/or screens is then supplied to a de-inking process. At the de-inking process, printing inks and any stickies still remaining are removed to increase the pulp's whiteness and purity. The de-inking process is usually a floatation process but may also include a washing process. More than one floatation processes may be used. Larger particles removed during the floatation process may be treated as waste and discharged to the waste treatment basin where it will settle as sludge.

During floatation de-inking, the pulp is fed into a large vat called a floatation cell where air is injected into the pulp. Collector chemicals may be introduced into the floatation cell to increase the efficiency of ink separation from the pulp. A typical floatation treatment might comprise one or more floatation cells, each of which is provided with a system to introduce air in the form of fine bubbles into the pulp slurry.

A collector chemical may be added to the pulp suspension prior to the floatation operation. The collector chemical may, for example, be a fatty acid soap or a non-ionic surfactant. The collector chemical attaches itself preferentially to the ink particles, rendering them hydrophobic, so that the ink particles have greater affinity for the air bubbles than for the water. The ink particles are thus lifted to the surface by the bubbles as they rise through the suspension to form a froth. The froth is collected and supplied to the settling basin. Ink particles having sizes in the range from about 20 microns to about 200 microns are concentrated in the froth removed from the top of the floatation cells, while a pulp suspension depleted in ink is withdrawn from the bottom.

The pulp suspension from the bottom of the floatation cells may then be mechanically cleaned, washed with water, and subsequently dewatered, to remove as much as possible of the remaining ink residue. The particles removed by cleaning and washing may be further treated by de-inking or added to the collective effluent stream.

The particles removed by the mechanical cleaning and the de-inking processes are subsequently removed from the effluent by screening and floatation and supplied to the settling basin to form sludge. The effluent formed during de-inking includes the initial froth from the floatation, the rejects from the mechanical separators, and the de-inked pulp wash water. This de-inking effluent forms Effluent 2. A waste paper mill processing approximately 250 tons of waste paper per day would produce approximately 0.2 mgpd of Effluent 2 having a BOD of approximately 500-1100 ppm.

The de-inked pulp, having a solids content of approximately 3 to 5 percent, is then provided to a paper making process. The pulp is diluted with clean water to form a very thin slurry. The clean water may be fresh water or recycled paper machine whitewater that brings the water to a pH of between about 6 and 9. The slurry is fed to a paper machine where it is first drained through a fine-mesh moving screen to bring the solids content to approximately 20 to 25 percent solids. The slurry is then introduced into a press section where more water is removed, and then onto a dryer section where the paper is run onto steam heated large drums to dry the paper. The paper is finally fed onto a winder where large paper rolls are formed. The paper making process reduces the slurry from a water content of at least about 95% to a water content of about 5%. More than one paper making process may be provided to form different types of paper.

Wash water from mechanical pressing and other water removed during the paper making process forms Effluent 3. Effluent 3 may be formed at about 0.2 mgpd in a waste paper mill processing approximately 250 tons of waste paper per day and have a BOD of about 500-1100 ppm.

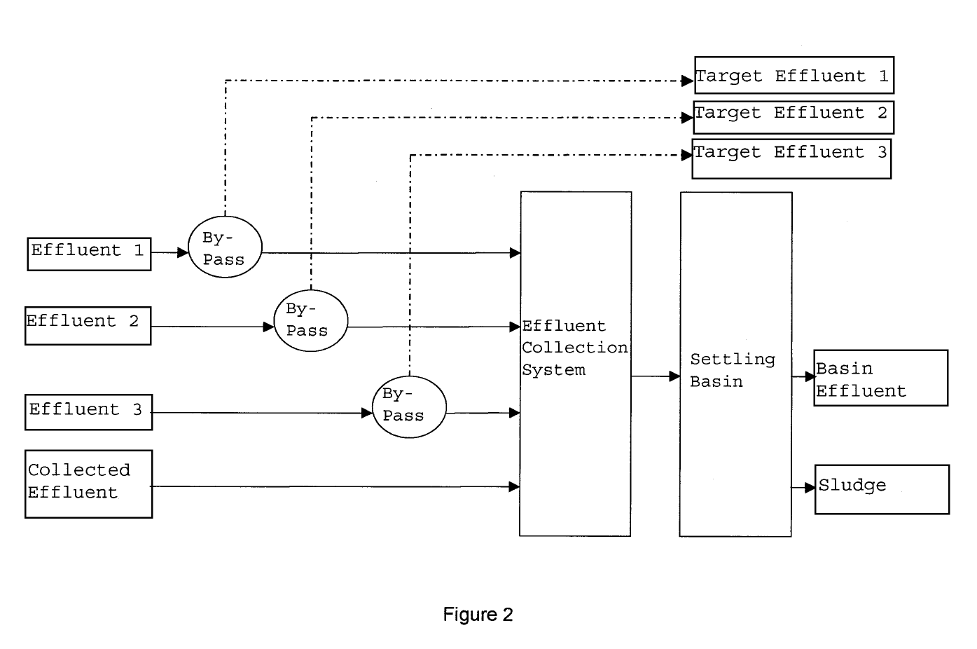

As shown in FIG. 2, the formed effluent streams 1, 2 and 3 may be provided to a effluent collection system or may be bypassed in whole or in part to be used as target effluent streams 1, 2 and 3. Effluent streams 1, 2 and 3 have a high BOD between about 500 ppm and 1100 ppm, and thus provide a high concentration of organic material including cellulose. Target effluent steams 1, 2 and 3 may be used in whole or in part for the production of ethanol. Target effluent streams 1, 2 and 3 have a BOD of between about 500 ppm and 1100 ppm. Additional waste paper mill effluent streams having high BOD levels may be additionally targeted in whole or in part. Furthermore, waste paper mill effluent streams having lower BODs may be targeted in order to provide additional organic material for ethanol production.

The remaining effluent created during various process steps in the waste paper process is provided to the effluent collection system as the collected effluent. The collected effluent streams forming the collective effluent stream have a BOD of typically between about 100 ppm and 1500 ppm, more typically between about 100 ppm and about 500 ppm. The collected effluent steam may be much less than about 500 ppm, and may be less than about 300 ppm. The collected effluent from the effluent collection system is provided to a settling basin. The collected effluent typically has a BOD of between about 200 ppm and about 300 ppm after final treatment in the settling basin. An effluent may be added to the collected effluent and not by-passed because of other conditions present in the effluent, such as low or high pH, even if the effluent has a high BOD.

The collected effluent is provided to a settling basin, typically a primary clarifier, to separate solids from the collected effluent. Additional separation steps may be provided, such as using drum screen separators, to further remove solids from the collected effluent. The solids separated from the collected effluent forms the waste paper mill sludge.

The sludge contains cellulose and waste paper mill solid byproducts including calcium carbonate and clay. The cellulose content of the sludge may range from about 50% to about 75%. The sludge is between about 30% and about 55% solids byproducts by weight.

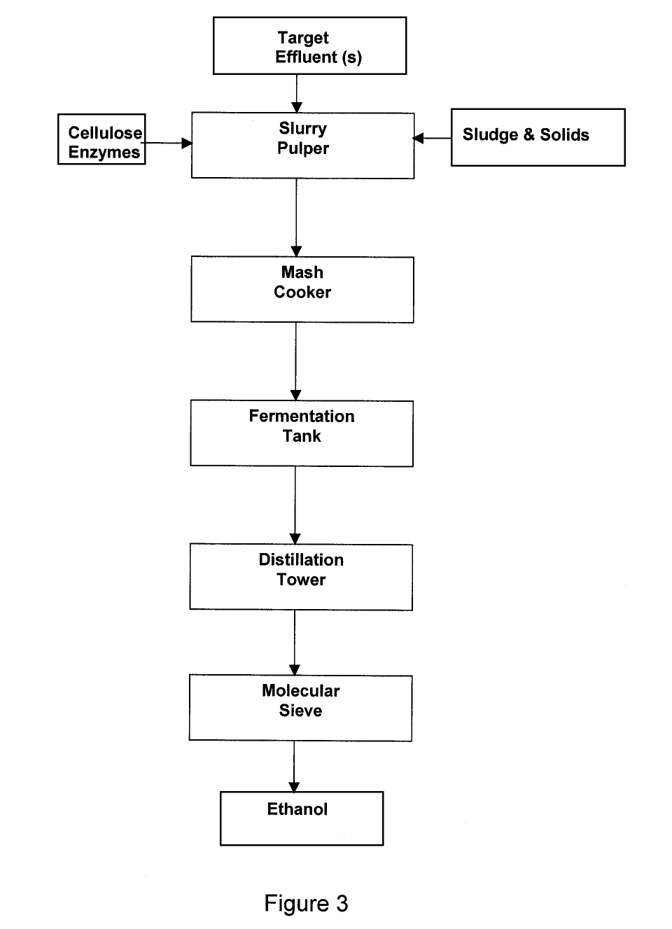

As shown in FIG. 3, target effluent(s) 1, 2 and 3 are used to form a slurry with the sludge from the settling basis. Target effluents 1, 2 and 3 may be used whole or in part to form the slurry. Additionally, one or more of the targeted effluents may not be added to the slurry. The amount of a target effluent used will depend upon the ethanol process input requirements, including overall BOD required, solids loading at the slurry pulper, and overall volume of each target effluent stream. Additional solids from other processes in the waste paper mill process may be added to the sludge. The sludge may also be combined with additional sources of cellulose including waste paper, paper processing sludge and other cellulose sources to increase cellulose content.

As shown in FIG. 3, the sludge is mixed with the target effluent stream(s) and cellulose enzymes at a slurry pulper to form a slurry. The slurry pulper has an agitator for mixing the sludge, effluent and enzymes and to further break down the cellulose of the sludge into separated fibers. Multiple pulpers may be used based on volume requirements or because of differing input sludge and solids characteristics. For example, a first pulper may be used for a sludge, and a second pulper may be used for a sludge and solids mixture.

An amount of ammonia may be provided to the slurry pulper through a reagent feed line to maintain the pH of the contents between about 4.5 and about 6.0 and to provide a nutrient to yeast that is used during later processing steps. It should be noted, that the reducing agent may be added to the reactor by any conventional chemical addition methods including manual addition. Solids from the sludge are substantially suspended in the effluent to form a slurry at the slurry pulper.

The enzymes may not be added at the pulper, but may be provided during distribution to the cooker reactors or at the cooker reactors. If enzymes are added at the slurry pulper, additional enzymes may be added during distribution to or at the reactors. A suitable enzyme is Trichoderma as provided Genencor International of Palo Alto, Calif.

A source of heat is applied to the reactors. The slurry, now referred to as a mash while in the reactors, is heated to between about 140° F. for between about 16 to about 24 hours to allow the cellulose enzymes to substantially convert the cellulose to sucrose. Any number of reactors may be used based on the size of the reactor and the amount of slurry to be processed.

The mash is heated until a solids portion and a liquid portion containing a substantial portion of sucrose are formed. The substantial conversion of cellulose to sucrose in the mash of greater than 98% to approximately 100% is possible. The sucrose is mainly contained in the liquid portion. The solids portion includes mainly ash and calcium carbonate. The solids portion is removed from the reactors by a solids removal line as is known in the art. The liquid portion is then filtered to remove any suspended solids and fed through a feed pipe to a fermentation tank. The liquid portion may be cooled before being fed to the fermentation tank, or may be allowed to cool upon entering the fermentation tank. The cooling may be performed by forced cooling or another known cooling method as is known in the art.

In the fermentation tank, a yeast is added to the liquid portion, which has been reduced to a temperature of between 80° F. and 90° F. The yeast may be a genetically manipulated yeast, as supplied by Genencor International of Palo Alto, Calif. After the yeast addition, the temperature of the liquid portion should be maintained to be between about 80° F. and 90° F. The fermentation tank is supplied with an agitator to circulate the yeast within the liquid portion.

The liquid portion within the fermentation tank is allowed to ferment until a desired substantial conversion of sucrose to ethanol is achieved. A conversion of approximately 98% to about 100% may be performed in about 48 hours. A gas outlet pipe may be used to remove carbon dioxide gas formed during the conversion of the sucrose to ethanol. The converted liquid portion containing ethanol is now referred to as a beer.

The beer is removed from the fermentation tank by a feed pipe and fed to a distillation tower. A distillation tower heat source supplies heat to the distillation tower to separate ethanol from the beer as is known in the art. The ethanol may still contain some water, but is preferably greater than about 90% ethanol. The remaining beer is removed from the bottom of the tower and further processed as waste. An ethanol steam is removed from the distillation tower and provided to a separator to remove water and purify the ethanol. The separator may be a molecular separator. The molecular separator may be a molecular sieve. The molecular separator separates water from the ethanol stream to produce a substantially pure ethanol product. After separation, the ethanol product is greater than about 99.99% ethanol. It should be understood that other alcohol and water separation techniques including distillation may be used depending upon the purity of the ethanol product desired or this purification step may be omitted or provided at a later time.

An exemplary waste paper mill producing approximately 2000 tons of paper per week, may produce approximately 300 tons per week of wet sludge of between about 30% to about 55% of solids. The solids contain ash and up to about 75% cellulose. The ash is mostly clay and calcium carbonate.

Such a mill may produce a total effluent stream of about 2.2 million gallons per day (gpd) having an average BOD of 320 ppm. The BOD is primarily from starch, glucose, and biological mass. The targeted effluent steams of such a mill may have a BOD of between about 500 ppm BOD to about 1100 ppm BOD and may be produced at about 300,000 gpd to about 500,000 gpd.

The targeted effluent streams would be mixed in whole or in part with the sludge to form a feed stream of about 5% to about 15% solids to be provided to ethanol production process. An ethanol facility having batch cooker reactors with a capacity of approximately 32,000 gallons, operating for between about 16 hours to about 24 hours per batch with appropriately selected enzymes, the facility further having appropriately sized fermentation tanks and distillation towers with a fermentation time of approximately 48 hours, and provided with a slurry of about 15% solids at between about 50,000 gpd to about 150,000 gpd, would produce between approximately 1 million to about 3 million gals per year of ethanol. Such a production system greatly reduces the amount of waste produced by a paper mill and generates a useful ethanol byproduct that may be used as a fuel.

While the invention has been described with reference to an example, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A method for producing ethanol, comprising:
providing an amount of waste paper process sludge;
combining the sludge with a selected waste paper process effluent stream to form a slurry containing cellulose;
adding a reducing agent to the slurry;
adding cellulose enzymes to the slurry;
cooking the slurry to convert a substantial portion of the cellulose to sucrose;
separating the cooked slurry into a liquid portion and a solids portion;
filtering the liquid portion to form a filtered liquid portion;
adding yeast to the filtered liquid portion;
fermenting the filtered liquid portion to form a beer whereby a substantial portion of the sucrose has been converted to ethanol;
distilling the beer to form an ethanol stream; and
providing the ethanol stream to a separator to remove water from the ethanol stream and form a substantially pure ethanol product.

2. The method of claim 1, wherein the selected waste paper process effluent stream comprises a de-inking system effluent.

3. The method of claim 2, wherein the selected waste paper process effluent stream further comprises whitewater from a paper machine process and starch losses.

4. The method of claim 3, wherein the selected waste paper process effluent stream has a BOD of between about 500 mg/l and about 1100 ppm.

5. The method of claim 1, wherein the substantially pure ethanol product is greater than 99.99% pure ethanol.

6. The method of claim 1, wherein the conversion of cellulose to sucrose is greater than 98%.

7. The method of claim 1, wherein the separator is a molecular sieve.

8. The method of claim 1, further comprising:
cooling the liquid portion before adding the yeast.

9. The method of claim 1, wherein the cooking takes place at a temperature of about 140° F. for between about 16 hours to about 24 hours.

10. The method of claim 1, wherein the fermenting takes place for about 48 hours.

11. A method for producing ethanol from waste paper mill sludge, comprising:
providing an amount of waste paper mill sludge;
combining the waste paper mill sludge with a selected paper mill effluent stream to form a slurry containing cellulose;
adding ammonia to the slurry;
adding cellulose enzymes to the slurry;
cooking the slurry at a temperature of about 140° F. for between about 16 hours to about 24 hours to convert a substantial portion of the cellulose to sucrose;
separating the cooked slurry into a liquid portion and a solids portion;
filtering the liquid portion;
adding yeast to the filtered liquid portion;
fermenting the filtered liquid portion to form a beer whereby a substantial portion of the sucrose has been converted to ethanol;
distilling the beer to form an ethanol stream; and
passing the ethanol stream through a molecular sieve to remove water from the ethanol stream and form substantially pure ethanol.

12. The method of claim 11, wherein the selected waste paper mill effluent stream has a BOD of between about 500 ppm and about 1100 ppm.

13. A system for producing ethanol, comprising:
a source of waste paper mill sludge containing cellulose;
a selected effluent stream containing dissolved organic compounds;
a reactor for receiving the sludge and the effluent stream and forming a slurry;
a source of cellulose enzymes for providing cellulose enzymes to the slurry;
a second reactor for receiving the slurry;
a heater for heating the second reactor to a temperature of about 140° F. for between about 16 hours and about 24 hours;
a solids removal line for removing solids from the second reactor;
a pipe connected to the second reactor for removing liquid from the second reactor;
a fermentation tank connected to the pipe for receiving the liquid removed from the second reactor;
a source of yeast for supplying yeast to the fermentation tank;
a second pipe connected to the fermentation tank for removing fermented liquid;
a distillation tower connected to the second pipe for receiving the fermented liquid from the fermentation tank;
a heat source for heating the distillation tower;
a third pipe connected to the distillation tower for removing an ethanol mixture from the distillation tower;
a molecular sieve attached to the third pipe for receiving the ethanol mixture; and
a pipe that removes a substantially pure ethanol product from the molecular sieve.

14. The system of claim 13, wherein the selected effluent stream comprises waste paper mill effluent having a BOD of between about 500 ppm and about 1100 ppm.

15. The system of claim 13, further comprising:
a second source of cellulose added to the reactor.

16. The system of claim 13, further comprising:
at least one other selected effluent stream containing dissolved solids added to the selected paper mill effluent stream.

17. The system of claim 16, further comprising:
wherein the at least one other selected effluent steam has a BOD of between about 100 ppm and 1500 ppm.
